(19) United States
(12) Patent Application Publication
(10) Pub. No.: US 2013/0020501 A1
(43) Pub. Date: Jan. 24, 2013

(54) UV Disinfecting Apparatus and System for Use With Contaminated Water

(71) Applicant: Jerry L. McKinney, Silsbee, IL (US)

(72) Inventor: Jerry L. McKinney, Silsbee, IL (US)

(21) Appl. No.: 13/189,148

(22) Filed: Jul. 22, 2011

(51) Int. Cl. A61L 2/10

(52) U.S. Cl. 250/437

(57) ABSTRACT

An apparatus for disinfecting contaminated water having a body forming a flow channel with a first end and a second end flowing generally from said first end of the flow channel to an outlet for removing contaminated water from the flow channel. There is a mount for suspending a UV bulb assembly above the channel at a distance above but sufficiently close to the level of contaminated water in the channel to effect UV radiation of contaminated water in the channel.

### FIELD OF THE INVENTION

The present invention relates to disinfecting contaminated water and, more specifically, disinfecting contaminated water using ultraviolet radiation.

### BACKGROUND OF THE INVENTION

Many homes and small businesses, particularly in rural and outlying areas which are not connected to rural treatment systems rely on septic tank systems, aerobic treatment systems, small package plants or other types of local treatment apparatus (collectively “packaged plants”) for wastewater treatment. These packaged plants generally use one or more tanks containing the treatment apparatus connected to a field drip system or drain field for ultimate disposal of the treated waters. Treatment entails degradation of waste in the water by the biological processes of bacteria and microorganisms. This “treated wastewater” generally contains bacteria and other microorganisms.

The fluid quality produced by packaged plants is usually regulated by one or more governmental agencies, most of which require that the discharged wastewater be disinfected to reduce the potential hazards caused by the bacteria/microbes in the treated effluent. One common type of disinfection treatment is chemical treatment with a chlorine type compound. Another common treatment is radiation with ultraviolet light (UV). What is known in the radiation of water with UV within the range from about 150 nm through about 300 nm is effective in destroying microorganisms. See for example U.S. Pat. No. 5,019,256; U.S. Pat. No. 5,256,299; U.S. Pat. No. 5,422,487; or U.S. Pat. No. 5,624,573. Prior art UV treatment systems are shown, for example, in U.S. Pat. Nos. 6,296,775, 7,250,610, 7,279,092, as well as U.S. Publication 2006/0163168, all of which are incorporated herein by reference for all purposes.

As evidenced by the patents and the prior art UV systems noted above, most UV disinfectant systems rely on flow of treated wastewater past a UV bulb which is positioned in an envelope, sleeve or the like of a material which is UV transparent. Typically these prior art UV disinfectant systems employ a quartz tube in which the UV bulb is disposed. However, the problem with a quartz housing or for that matter a housing over the UV bulb made of any type of UV transparent material is that over time a film of scum, particulates or the like forms on the chamber or sleeve and reduces the amount of UV radiation passing through to the point where the treated wastewater is not effectively disinfected.

In addition to treated wastewater there also exists a problem in disinfecting contaminated water from other sources. In many areas of the world infrastructure which provides potable water for human use, e.g., consumption, baths, etc. is inadequate. For example, in many parts of the world while municipal water may be delivered to residential or commercial sites, the pumping systems do not produce sufficient water pressure to service all of the household needs of the numerous residences attached or connected to the municipal system. In many instances, it is common to place a holding tank on the elevated portion of the residence, e.g., the roof, pump the water into the holding tank, and then use the head pressure of the water at the elevated location to accommodate normal household needs such as showers, faucets, etc. Still another problem with municipal water in these somewhat underdeveloped locations is that the water may become contaminated due to inadequate chlorination, leaking pipes, etc. Accordingly, even though the water is from a municipal system, it is still contaminated and can cause serious illness. Lastly, since the water in these environments is in the holding tanks as described above and may be quiescent for undesirable periods of time, there is a chance for the growth of microorganisms, bacteria and the like which would again contaminate the water prior to use.

### SUMMARY OF THE INVENTION

In a general aspect, the present invention provides a UV disinfection apparatus for contaminated water wherein the UV light assembly is not in contact with the contaminated water.

In yet another aspect, the present invention provides a UV disinfection system comprising a tank or vessel for holding contaminated water and UV disinfection apparatus for disinfecting the contaminated water wherein the UV light assembly is not in contact with the contaminated water.

In one aspect the present invention provides a UV disinfection apparatus for contaminated water wherein the UV light assembly is not in contact with the treated wastewater.

In another aspect the present invention provides a UV disinfection apparatus for contaminated water which avoids deposition of deposits on the UV bulb of a UV disinfectant apparatus.

In still another aspect the present invention provides a UV disinfection system having a tank for holding contaminated water and a UV disinfection apparatus for disinfecting the contaminated water wherein the UV light assembly is not in contact with the contaminated water.

In still a further aspect of the present invention there is provided a UV disinfection system comprising a tank for contaminated water and a UV disinfection apparatus which avoids deposition of deposits on the UV bulb of the UV disinfection apparatus.

In yet a further aspect of the present invention there is provided a system for disinfecting contaminated water, such as described above which is used in conjunction with an alternative energy source to supply electric power to operate the UV bulb as well as any pumps.

These and further features and advantages of the present invention will become apparent from the following detailed description, wherein reference is made to the figures in the accompanying drawings.

### BRIEF DESCRIPTION OF THE DRAWINGS

FIG. 1 is an elevational view, partly in section, showing one embodiment of the UV disinfectant system of the present invention.

FIG. 2 is a view similar to FIG. 1 showing another embodiment of the UV disinfectant system of the present invention.

FIG. 3 is an elevational view, partly in section, showing a UV disinfectant apparatus of the present invention.

FIG. 4 is a cross-sectional view taken along the lines 4-4 of FIG. 3.

FIG. 5 is a view similar to FIG. 4 showing another embodiment of the UV disinfectant apparatus of the present invention.

FIG. 6 is an elevational view similar to FIG. 1 showing another embodiment of the UV disinfectant system of the present invention.

FIG. 7 is an elevational view, partly in section, of yet another embodiment of the UV disinfectant apparatus of the present invention.

FIG. 8 is a cross-sectional view taken along the lines 8-8 of FIG. 7.

FIG. 9 is an elevational view, partly in section, of another embodiment of the UV disinfectant system of the present invention.

FIG. 10 is an elevational view showing the disinfectant system of the present invention in conjunction with a solar panel to supply alternative energy to the electrical components of the UV disinfectant system.

FIG. 11 is a view taken along the lines 11-11 of FIG. 10.

FIG. 12 is another view, similar to FIG. 10 showing the UV disinfectant system of the present invention in conjunction with a wind power turbine.

### DETAILED DESCRIPTION OF PREFERRED EMBODIMENTS

As used herein, the term “treated wastewater” generally refers to wastewater from a septic system which has been subjected to a pretreatment or settling tank to remove large solids and aerobic digestion to produce substantially clarified, treated wastewater. The term “contaminated water” refers to treated wastewater as well as water from other sources which is intended to be used as potable water or at the very least relatively bacteria and microorganism free water. Thus, in the description which follows, treated wastewater and contaminated water may be used inter-changeably.

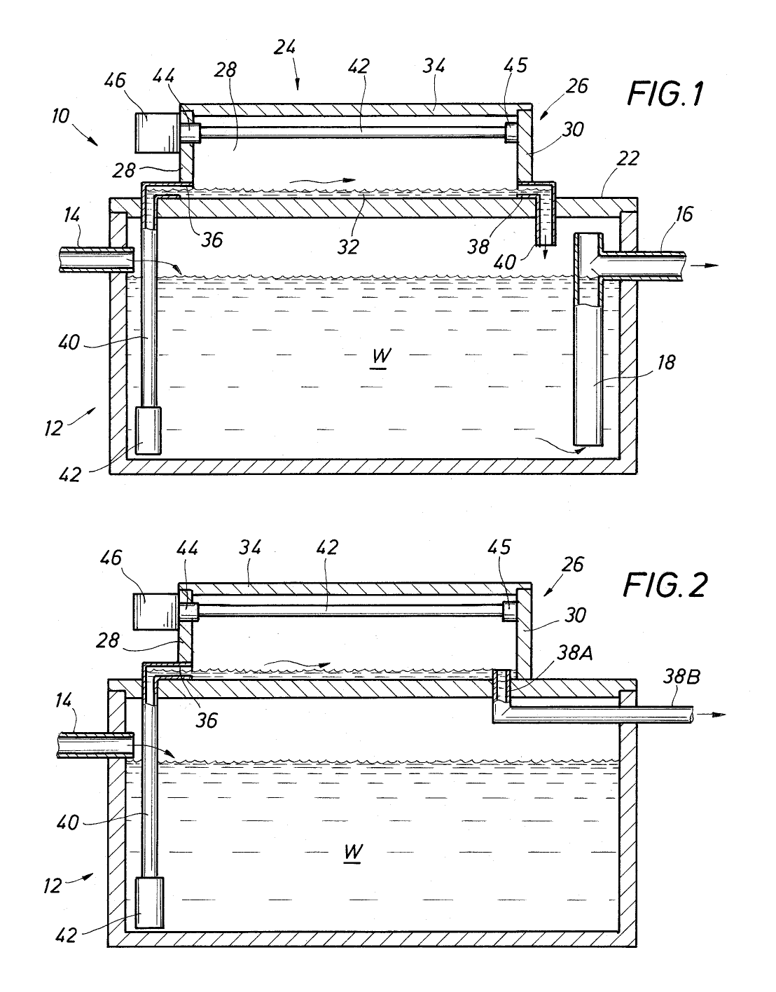

Referring to FIG. 1, there is shown an embodiment of the UV disinfectant system, indicated generally as 10. The embodiment shown in FIG. 1 comprises a tank 12 having a volume W of treated wastewater therein. Tank 12 has an inlet 14 and an outlet 16, inlet 14 being connected to a source of clarified treated wastewater from a packaged plant, outlet 16 being connected to a riser pipe 18 extending into treated wastewater W for gravity flow of treated wastewater W out of outlet 16 of tank 12. Although not shown, typically tank 12 has a cover with a neck portion and an access hatch which can be removed and the interior of tank 12 accessed for maintenance or the like.

The UV disinfectant system 10 comprises a UV disinfectant apparatus shown generally as 24 comprising a housing shown generally as 26 having first and second end walls 28 and 30, respectively, a bottom wall 32 and first and second side walls (not shown). A removable cover 34 cooperates with housing 26 to form a chamber 28. Housing 26 has an inlet 36 located generally at first end wall 28 and an outlet 38 located generally at second end wall 30. As seen, inlet 36 is connected via a pipe 40 to a pump 42 disposed in tank or vessel 12 for pumping treated wastewater W into inlet 36 of housing 26. The bottom wall 32 of housing 26 forms a flow channel along which treated wastewater pumped via pump 42 flows toward the outlet 38 and via a down corner 40 back into tank 12.

An elongate UV bulb 42 extends between mounts 44 and 45 and first and second side walls 28 and 30, respectively. An electrical component box 46 is connected to a suitable source of power and houses necessary electronics, e.g., alarms, etc. for operating UV bulb 42. As seen, UV bulb 42 and the channel formed by bottom wall 32 are generally elongate and parallel to one another. UV bulb 42 is spaced above the level of treated wastewater W in chamber 28. It will be appreciated, that bulb 42 will be spaced above the level of the treated wastewater in housing 28, but sufficiently close to the level of treated wastewater in chamber 28 such that UV light from bulb 42 effectively irradiates the treated wastewater W flowing through housing 26 from inlet 36 to outlet 38. The spacing between UV bulb 42 and the level of treated wastewater W in chamber 28 will depend upon the size and strength of UV bulb 42. In operation, and with pump 42 activated, treated wastewater W is pumped out of tank 12 through housing 26 and returned to tank 12 via down corner 40. This constant recirculation of treated wastewater W through chamber 28 ensures the most effective UV irradiation of the treated wastewater W prior to it being discharged by gravity via riser 18 and outlet 16. In this regard it is known that in many UV disinfectant systems for treating wastewater, the wastewater generally makes a maximum of two passes past the UV bulb prior to being discharged. This is not always effective for the maximum kill rate of bacteria and microorganisms.

Since UV bulb 42 is spaced from the level of treated wastewater W in chamber 28, and unlike many prior art UV disinfectant systems, UV bulb 42 does not accumulate a film from scum, particulates or the like which are present in the treated wastewater W. Not only does this enhance the level of treatment but provides for more maintenance free operation. In the latter regard, many prior art UV disinfectant systems have the UV bulb in a substantially UV transparent envelope, sleeve or the like, the UV bulb being sealed from the treated wastewater by the sleeve. Because the sleeve is in direct contact with the treated wastewater W, it is prone to the build up of scum, particulates or the like which degrades the strength of the UV radiation reaching the treated wastewater. Accordingly, it is necessary to periodically remove the UV disinfectant system and clean the sleeve. Further, since even a quartz envelope, sleeve or the like can attenuate the UV radiation to some extent, by using the present invention either a weaker bulb can be employed or if a bulb typically used in the prior art UV disinfectant systems is employed, the treated wastewater W will receive greater UV irradiation.

Referring now to FIG. 2, there is shown a slightly different embodiment of the UV disinfectant system shown in FIG. 1. The system shown in FIG. 2 differs from that shown in FIG. 1 in that rather than the treated, UV irradiated wastewater being returned to the interior of tank 12, it flows through an outlet 38A and a discharge pipe 38B. In other words, unlike the system shown in FIG. 1 where there is recirculation of treated wastewater W past the UV bulb 42, in the embodiment shown in FIG. 2 there is a one-time pass of the treated wastewater from tank 12. Although this one time pass is not desirable for reasons mentioned above, in systems where only small discharges of wastewater are occurring, i.e., from a residence of the like where only wastewater from a toilet (about 1.6 gallons) is being handled, this one time pass can be effective. However, as will be seen hereafter in cases where there are large charges of wastewater to this packaged system, multiple passes past the UV bulb provided by the present invention are much preferred.

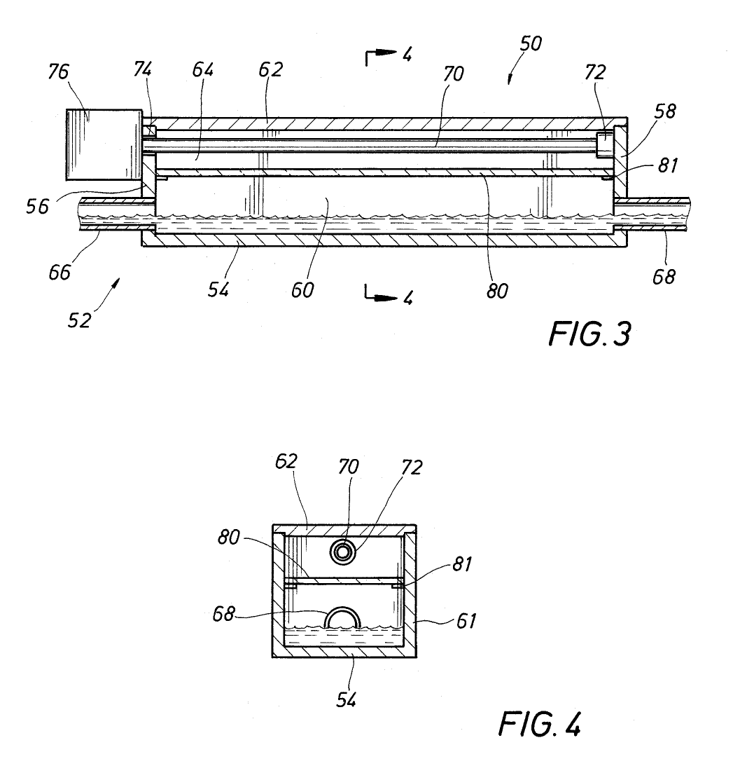

Referring now to FIGS. 3 and 4, there is shown an embodiment of the UV disinfectant apparatus of the present invention. The apparatus shown generally as 50 comprises a housing shown generally as 52 having a bottom wall 54, a first end wall 56 and a second, spaced end wall 58. Housing 52 further comprises a first end wall 60 and a second end wall 61. A removable cover 62 in cooperation with end walls 56, 58, bottom wall 54, and the side wall forms a chamber 64. There is an inlet 66 through end wall 56 and an outlet 68 through end wall 58. Bottom wall 54 in conjunction with portions of end walls 56, 58 and side walls 60, 61 forms a flow channel through which treated wastewater introduced via inlet 66 flows through chamber 64 and out outlet 68.

Disposed in chamber 64 is a UV bulb 70, UV bulb 70 being connected by a socket 72 to end wall 58 and extending through an aperture 74 in end wall 56 into an electrical component box 76 containing electric power leads, alarms, etc. customarily used to operate UV bulb 70.

Optionally, although not necessary, there is a UV guard or splash plate 80 disposed between UV bulb 70 and the level of treated wastewater W in chamber 64, plate 80 resting on a peripheral ledge formed on the walls of housing 52. Splash plate or guard 80 serves the purpose of preventing any unexpected surges of treated wastewater W in chamber 64 from contacting UV bulb 70.

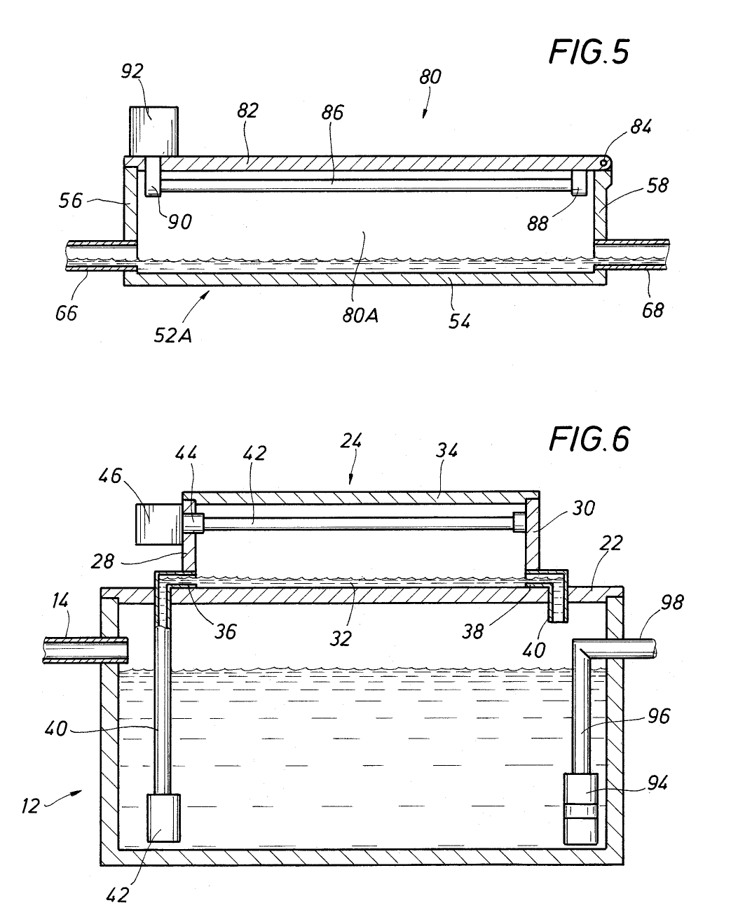

Referring now to FIG. 5, there is shown another embodiment of the UV disinfectant apparatus of the present invention. The UV disinfecting apparatus of FIG. 5, shown generally as 80 comprises a housing 52A having structural features much in the manner described like those in FIG. 3. However, in the embodiment shown in FIG. 5, there is a hinged lid 82 hinged at 84 to end wall 58. UV bulb 86 is mounted in sockets 88 and 90 which are attached to lid 82. Socket 90 is in turn electrically connected to an electrical control box 92 which houses electronics, power, etc. for operation of bulb 86. Since lid 82 is hinged, to access the chamber 80A in which UV bulb 86 is disposed, it is only necessary to raise lid 82 and rotate it around hinge 84. This will allow full access to chamber 80A and allow replacement of bulb 86 or other maintenance required. Although not shown, it will be appreciated that lid 82 and end wall 56 could be provided with switch contacts which, when lid 82 is raised, will disconnect power to bulb 86 thereby preventing a maintenance worker or the like from having his/her eyes exposed to UV radiation from bulb 86.

Turning now to FIG. 6, there is shown another embodiment of the UV disinfecting system of the present invention. The embodiment shown in FIG. 6 is very similar to that shown in FIG. 1 with the exception that rather than relying on gravity flow of treated wastewater W out of tank 12, there is a second pump 94 disposed in treated wastewater W which pumps treated, irradiated wastewater through riser pipe 96 and outlet 98 for disposal.

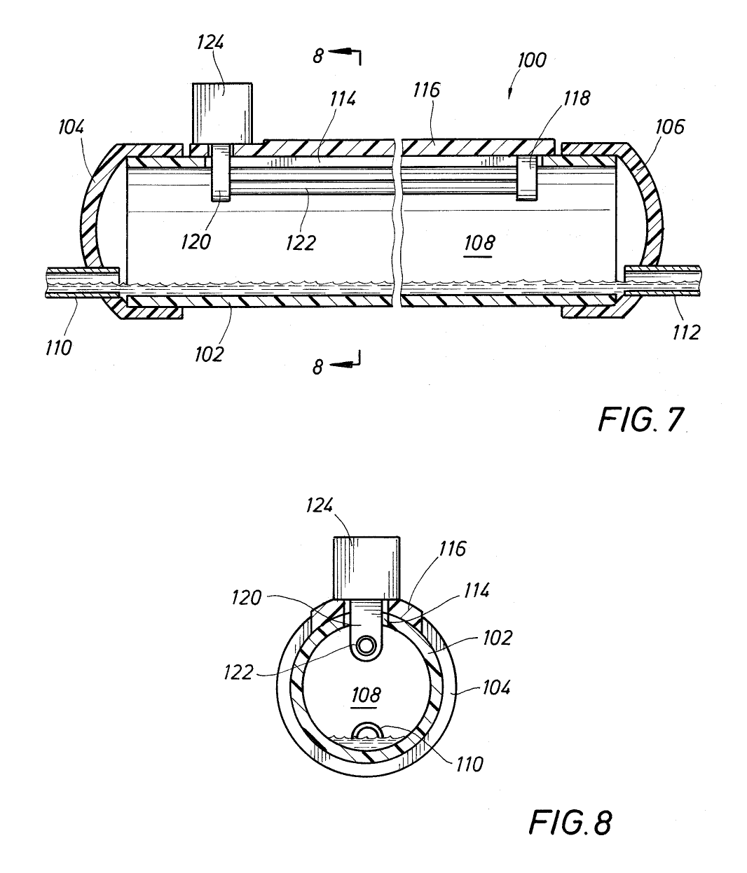

Referring to FIG. 7, there is shown yet another embodiment of the UV disinfecting apparatus of the present invention. In the embodiment shown in FIG. 7, the UV disinfecting apparatus shown generally as 100 comprises a cylindrical tube 102, e.g., a PVC tube, provided with end caps 104 and 106. There thus is formed a chamber 108, in the interior of tube 102. An inlet 110 is used to introduce treated wastewater via pump, airlift, or other such pump system into chamber 108, the treated wastewater flowing lengthwise through tube 108 to outlet 112 whereby it will be recycled to the holding tank for the treated wastewater for further UV treatment or sent to disposal. Because of the cylindrical nature of tube 102, and as best seen in FIG. 8, a contoured flow channel is formed.

Tube 102 has a slot 114 generally diametrically opposite the channel for treated wastewater formed in tube 102. Accordingly, in use, apparatus 100 would be oriented with slot 114 up and the bottom of tube 102 down. Overlying slot 114 is a cover 116, cover 116 being a segment of a plastic, e.g., PVC pipe which has an ID substantially equal to the OD of tube 102. Attached to cover 116 are a pair of spaced brackets 118 and 120 which carry sockets, only one of which (socket 120) is shown in FIG. 8. Received in the sockets of the brackets 118 and 120 is UV bulb 122. An electrical component box 124 carried by cover 116 contains power lines, alarms, etc. used to operate UV bulb 122. As seen in FIG. 8, slot 114 has a width slightly larger than the width of brackets 118 and 120 such that when the cover 114 carrying the brackets 118 and 120 and electronic component box 124 is disposed on tube 102, the UV bulb 122 extends into chamber 108 and remains oriented substantially vertically above the flow channel formed at the lowermost portion of tube 102.

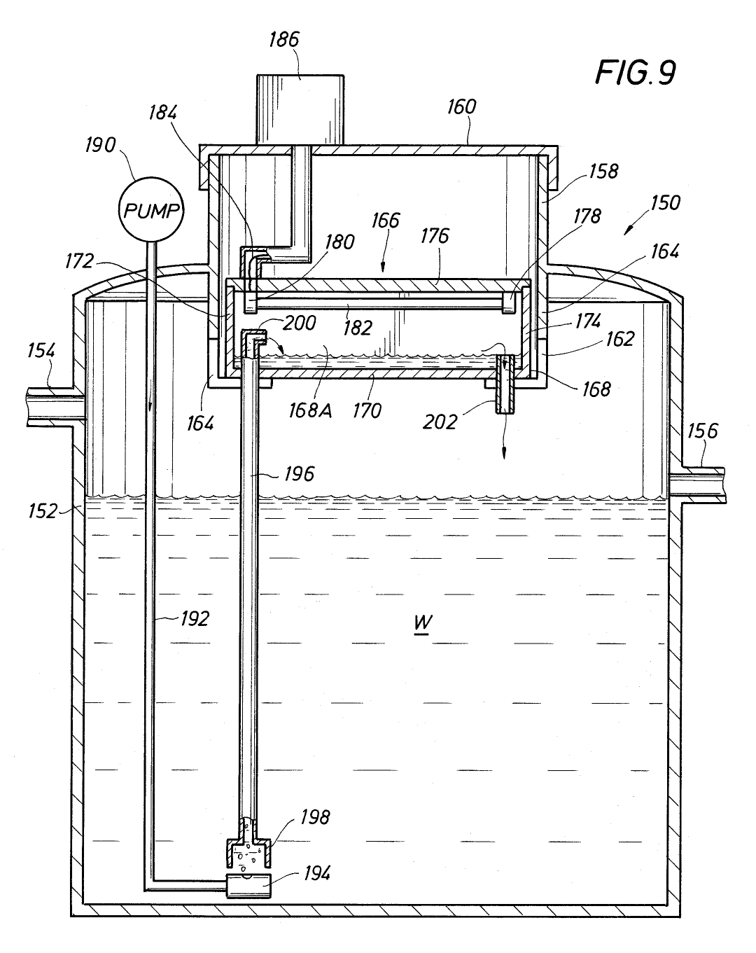

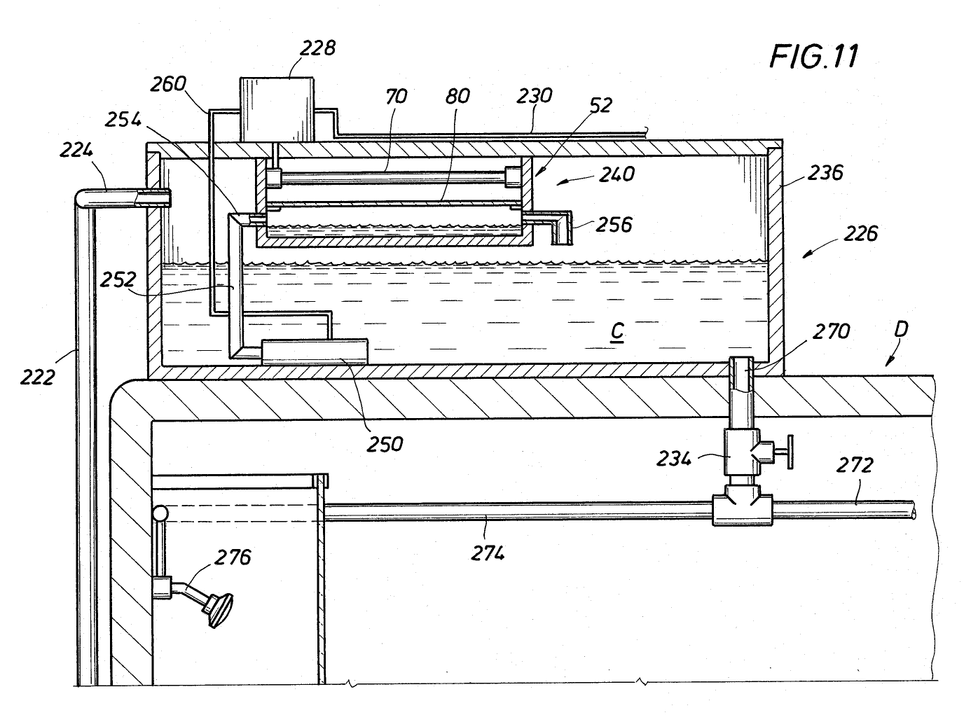

Referring now to FIG. 9 there is shown another embodiment of the UV disinfectant system of the present invention. The UV disinfectant system of FIG. 9, shown generally as 150 comprises a pump or holding tank 152 having an inlet 154 and an outlet 156. Treated wastewater W enters pump or holding vessel 152 through inlet 154 and leaves it via gravity through outlet 156. It will be understood, and well known to those skilled in the art that vessel 152 could be supplied with a pump such as shown in FIG. 6 to pump treated wastewater W out of tank 152.

Tank 152 has a neck portion 158 with a removable cover 160 whereby the interior of vessel 152 can be accessed as necessary. A pair of L-shaped mounting brackets 162 and 164 are attached in a suitable manner to a cylindrical extension 164 of neck portion 158. Brackets 162 and 164 serve to hold a UV disinfecting apparatus shown generally as 166 and comprising a housing or body, generally rectangular in shape, 168 which has a bottom wall 170 forming a flow channel extending from a first end wall 172 of housing 168 to a second end wall 174 of housing 168. Housing 168 is provided with a removable cover 176. Attached to the underside of cover 176 are sockets 178 and 180 in which is received a UV bulb 182. Electric power lines 184 extend through an electronic component box 186 mounted on the top of cover 160. The air lift pump comprises a compressor or the like 190 which pumps air through a line 192 to a gas sparger 194 located between a tube 196 having an intake 198 and a gas sparger 194. Tube 196 has an L-shaped top 200 opening into the chamber 168A formed in housing 168. In operation, and while pump or compressor 190 is running, air is pumped through line 192 and sparger 194 and, as it bubbles upward through mouth 198 of tube 196, it lifts treated wastewater W, in a well-known manner, through outlet 200 into UV chamber 168A. The treated wastewater flowing through chamber 168A is irradiated by UV bulb 182 and exits housing 168 through a down corner 202 where it is returned to the treated wastewater W in vessel 152. Again, there is, so long as pump 190 is in operation, continuous UV irradiation of the treated wastewater W in vessel 152. In this regard, and with respect to the other embodiments discussed above, regardless of the pump system employed, the treated wastewater W can be passed through the UV treatment apparatus 166 as often as desired, i.e, pump 190 could be set to run continuously.

One of the many advantages of the UV disinfectant apparatus and system of the present invention, over and above its ability to ensure greater disinfection of treated wastewater is the ability to ensure that this enhanced disinfectant treatment occurs regardless of the load placed on the system. For example, in the typical residential environment, a toilet flush will send about 1.6 gallons of water to the septic system, e.g., an aerobic wastewater treatment system. In prior art systems where there is a maximum of about two passes of treated wastewater past the UV bulb prior to disposal, this relatively small amount of water from a toilet flush can be fairly effectively disinfected by the prior art systems. However, in cases where there is a large charge of wastewater, e.g., from an emptying bath tub, washing machine or the like, the rate of discharge of water through the prior art UV disinfecting systems is too great to allow ample irradiation. However, using the apparatus or system of the present invention wherein the treated wastewater can be passed multiple times, e.g., as long a pump system is designed to run, a greater kill rate of bacteria and microbes is achieved.

In the description above, reference is made to a pump, e.g., pump 42 for introducing treated wastewater from tank 12 into UV disinfecting apparatus 24. It will be understood that the term “pump system” as used herein refers not only to a submersible pump, of whatever type, but also to gas lift systems which can be used to introduce treated wastewater W from tank 12 into the UV disinfecting apparatus, whether the UV disinfecting apparatus is disposed interiorly of the tank or exteriorly thereof.

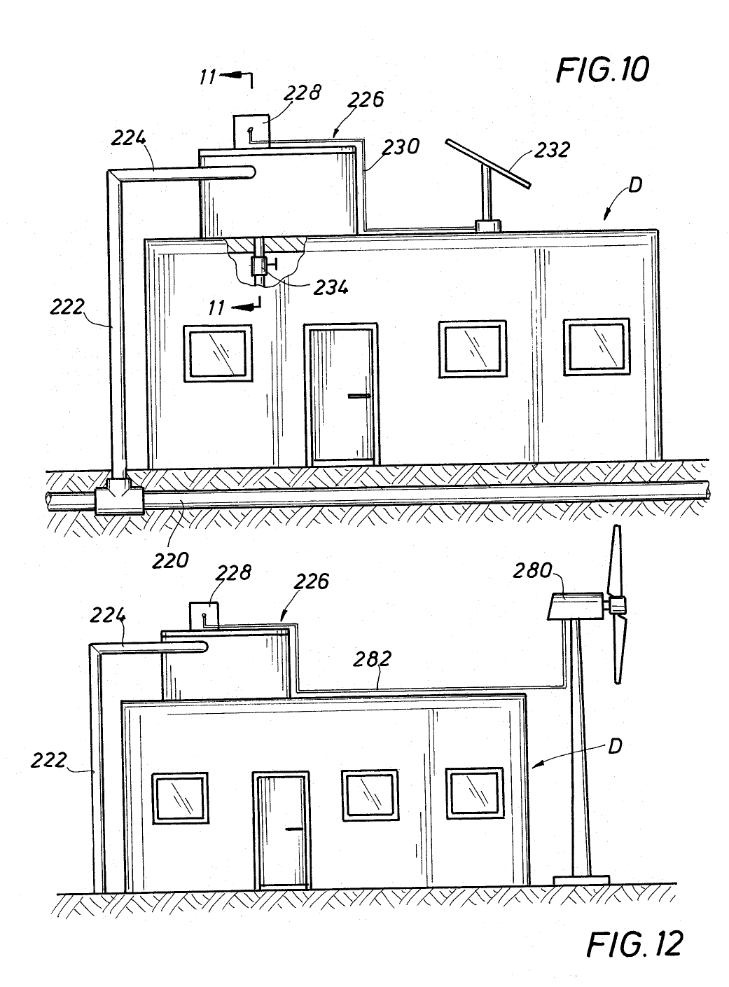

Referring now to FIGS. 10-12, there is shown the disinfectant system of the present invention used for producing potable water or at least water with a vastly reduced bacteria and microorganism content sufficient for at least some forms of human usage. Referring first to FIG. 10, there is shown a dwelling D which could be a residence or business but in any event, is suitable for human habitation. A main water supply 220 which can be a municipal source or other generally centralized source of water is connected to a riser 222 having a branch 224 which feeds water from main line 220 into a holding tank of a UV disinfectant system shown generally as 226. Disinfectant system 226 can comprise any of the systems/apparatus discussed above in various other modified embodiments, particularly the use of the UV disinfectant system/apparatus as discussed above wherein there is recycled UV irradiated contaminated water through the UV disinfectant system/apparatus. An electronic component box 228, similar to the electronic component boxes discussed above with respect to the other embodiments, is connected by power line 230 to a solar panel or array of solar panels 242 for supplying electric power to electronic component box 228. The embodiment of FIG. 10 is shown in greater detail in FIG. 11 which shows a main valve 234 through which disinfected water from holding tank in disinfectant system 226 flows into the interior of dwelling D.

Referring now to FIG. 11 there is shown in greater detail the embodiment depicted in FIG. 10. The UV disinfectant system 226 comprises a holding tank or vessel 236 into which contaminated water C flows, via branch 224, to riser 222 and main line 220. A UV disinfectant apparatus shown generally as 240 is mounted in tank 236, unit 240 being similar to that shown in FIG. 3 in the sense that there is a quartz splash plate 280 as shown in FIG. 3 between UV bulb 70 and the level of water in the housing 52. UV bulb 70 is connected to electronic component box 228 and thereby power is supplied to UV bulb 70. A submersible pump 250 is disposed in vessel 236 and pumps water through a riser 252 into the inlet 254 into the interior of the housing 52. UV irradiated water flows out of housing 52 via outlet 256 and back into vessel 236 such that it is continuously recycled through the UV disinfectant apparatus 240. The power line 230 and the solar panel 232 also supplies power via power line 260 to submersible pump 250. An outlet 270 from vessel 236 is connected to valve 234 and hence to the internal piping or plumbing 272, 274, in dwelling D pipe 274 leading to a showerhead 276 accessible to dwellers in dwelling D. It will also be appreciated that the piping or plumbing in dwelling D could also go to faucets, bath tubs or other user accessible fixtures in dwelling D.

Turning now to the embodiment shown in FIG. 12, it is substantially the same as that shown in FIGS. 10 and 11 with the exception that instead of the solar panel array 232, electric power is supplied by virtue of a wind turbine 280 via a power line 282.

It will be appreciated that UV disinfectant system 226 need not be disposed at an elevated position relative to a user accessible outlet, e.g., a shower, faucet, bath tub or the like. Indeed, much like the embodiment shown in FIG. 6, the UV disinfecting unit 226 can be supplied with a second pump such as pump 94 which would supply water to the main valve 234 thereby obviating the necessity for the disinfectant unit 226 to be mounted at an elevated position relative to a user accessible outlet.

The term “alternative energy source” refers to an energy source which is not dependent on the burning of fossil fuel, solar power and wind power being prime examples. Further, as is well known, solar panels and smaller wind turbines can be installed near the UV disinfectant system of the present invention and be small enough to only supply to the electronic components of the UV disinfectant system, and/or on a slightly larger scale to the dwellings where the UV disinfectant system is located.

One of the features of the present invention is that because of recycling water past the UV bulb assembly, a smaller UV bulb can be employed minimizing the power needs of that component.

Although specific embodiments of the invention have been described herein in some detail, this has been done solely for the purposes of explaining the various aspects of the invention, and is not intended to limit the scope of the invention as defined in the claims which follow. Those skilled in the art will understand that the embodiment shown and described is exemplary, and various other substitutions, alterations and modifications, including but not limited to those design alternatives specifically discussed herein, may be made in the practice of the invention without departing from its scope.

1. An apparatus for disinfecting contaminated water comprising:
a body forming a flow channel, said flow channel having a first end, a second end, and an outlet generally near said second end, for removing contaminated water passing through said flow channel generally from said first end, said body comprising a housing having a removable top disposed above said flow channel; and
a UV bulb mounted to said top and above said channel at a distance above but sufficiently close to the level of contaminated water in said channel to effect UV radiation of contaminated water in said channel.

2. (canceled)

3. The apparatus of claim 1, wherein said top is swing hinged to said body.

4. (canceled)

5. The apparatus of claim 1, wherein said flow channel is generally elongate.

6. The apparatus of claim 5, wherein said UV bulb is generally elongate, the long axis of said flow channel and the long axis of said UV bulb being generally parallel to one another.

7. The apparatus of claim 1, wherein there is a UV transparent shield between said UV bulb and said channel.

8. The apparatus of claim 1, wherein said body comprises a generally cylindrical housing, said cylindrical housing having a slot extending generally lengthwise of said cylindrical housing and said UV bulb is received through said slot.

9. The apparatus of claim 8, wherein said UV bulb is attached to a removable cover overlying said slot.

10. A system for disinfecting contaminated water comprising:
a tank for holding contaminated water;
a UV disinfecting apparatus comprising
a body forming a flow channel, said flow channel having a first end, a second end, and an outlet, generally near said second end, for removing contaminated water passing through said flow channel generally from said first end;
a mount for suspending a UV bulb above said channel at a distance above but sufficiently close to the level of contaminated water in said channel to effect UV radiation of contaminated water in said channel, said contaminated water being in said tank below said UV disinfecting apparatus; and
a pump system for pumping contaminated water into said flow channel from said tank through said flow channel and back to said tank in a recirculating manner to provide UV treated water.

11-12. (canceled)

13. The system of claim 10, wherein said tank has an outlet for gravity flow of UV treated water out of said tank.

14. The system of claim 10, wherein there is a second pump in said tank for pumping UV treated water out of said tank.

15. The system of claim 10, wherein said apparatus is disposed in said tank.

16. The system of claim 10, wherein said pump system comprises a submersible pump.

17. The system of claim 10, wherein said pump system comprises a gas lift.

18. A system for disinfecting contaminated water comprising:
a tank for holding contaminated water;
a UV disinfecting apparatus comprising:
a body forming a flow channel, said flow channel having a first end, a second end, and an outlet, generally near said second end, for removing contaminated water passing through said flow channel, generally from said first end;
a mount for suspending a UV bulb above said channel at a distance above but sufficiently close to the level of contaminated water in said channel to effect UV radiation of contaminated water in said channel, said contaminated water being in said tank below said UV disinfecting apparatus;
a pump system for introducing contaminated water into said flow channel and circulating said contaminated water from said tank through said flow channel and back to said tank to provide UV treated water; and
an alternative energy source positionable in the vicinity of said system for supplying electric power to at least one of said UV bulb and said pump system.

19. (canceled)

20. The system of claim 18, wherein said tank has an outlet for gravity flow of UV treated water out of said tank.

21. The system of claim 18, wherein there is a second pump in said tank for pumping UV treated water out of said tank.

22. The system of claim 21, wherein said second pump is powered by said alternative energy source.

23. The system of claim 18, wherein said apparatus is disposed in said tank.

24. The system of claim 18, wherein said pump system comprises a submersible pump.

25. The system of claim 18, wherein said pump system comprises a gas lift.

26. The system of claim 18, wherein said tank, said apparatus, and said pump system are positioned at an elevated position relative to a user accessible outlet below said elevated position.

27. The system of claim 18, wherein said alternative energy source is one of solar energy or wind energy.
